SEQUENCE LISTING (14 sequences)

SEQ ID NO: 1 - DNA, 22 bp, Artificial Sequence
  IFN-gamma 5′ Primer
tggctgtttctggctgttactg

SEQ ID NO: 2 - DNA, 22 bp, Artificial Sequence
  IFN-gamma 3′ Primer
acgcttatgttgttgctgatgg

SEQ ID NO: 3 - DNA, 22 bp, Artificial Sequence
  TNF-alpha 5′ Primer
ccagaccctcacactcagatca

SEQ ID NO: 4 - DNA, 21 bp, Aritificial Sequence
  TNF-alpha 3′ Primer
cacttggtggtttgctacgac

SEQ ID NO: 5 - DNA, 20 bp, Artificial Sequence
  IL-12/23p40 5′ Primer
ggaagcacggcagcagaata

SEQ ID NO: 6 - DNA, 25 bp, Artificial Sequence
  IL-12/23p40 3′ Primer
gaacttgagggagaagtaggaatgg

SEQ ID NO: 7 - DNA, 20 bp, Artificial Sequence
  IL-10 5′ Primer
gaagcatggcccagaaatca

SEQ ID NO: 8 - DNA, 20 bp, Artificial Sequence
  IL-10 3′ Primer
tgctccactgccttgctctt

SEQ ID NO: 9 - DNA, 20 bp, Artificial Sequence
  IDO-1 5′ Primer
gtgggcagcttttcaacttc

SEQ ID NO: 10 - DNA, 20 bp, Artificial Sequence
  IDO-1 3′ Primer
gggctttgctctaccacatc

SEQ ID NO: 11 - DNA, 24 bp, Artificial Sequence
  IDO-2 5′ Primer
tgcctgatggcctataaccagtgt

SEQ ID NO: 12 - DNA, 24 bp, Artificial Sequence
  IDO-2 3′ Primer
tgcaggatgtgaacctctaacgct

SEQ ID NO: 13 - DNA, 20 bp, Artificial Sequence
  beta-actin 5′ Primer
ggcccagagcaagagaggta

SEQ ID NO: 14 - DNA, 20 bp, Artificial Sequence
  beta-actin 3′ Primer
ggttggccttagggttcagg

(19) United States
(12) Patent Application Publication
(10) Pub. No.: US 2010/0261774 A1
(43) Pub. Date: Oct. 14, 2010

(54) Methods for the modulation of Leishmania major infection in mammals

(75) Inventors: Christopher L. Karp, Cincinnati, OH (US); Senad Divanovic, Cincinnati, OH (US)

Correspondence Address: DAVIS WRIGHT TREMAINE LLP - San Francisco, 505 MONTGOMERY STREET, SUITE 800, SAN FRANCISCO, CA, 94111, US

(73) Assignee: Children's Hospital Medical Center, Cincinnati, OH (US)

(21) Appl. No.: 12/658,895

(22) Filed: Feb. 16, 2010

Related U.S. Application Data
(60) Provisional application No. 61/152,491, filed on Feb. 13, 2009.

(51) Int. Cl. A61K 31/405; A61P 33/02

(52) U.S. Cl. 514/419

(57) ABSTRACT

Embodiments of the invention include methods for treating an infection of a mammal by an organism, such as Leishmania major by administering an effective amount of an inhibitor of indolamine 2,3-dexygenase (IDO). A preferred inhibitor is 1-methyl-tryptophan (1MT). The treatment can be highly beneficial as a treatment for visceral leishmaniasis.

### CROSS REFERENCE TO RELATED APPLICATIONS

This application claims the benefit of priority under 35 U.S.C. §119(e) to U.S. Provisional Application No. 61/152,491, filed on Feb. 13, 2009, entitled METHODS FOR MODULATION OF LEISHMANIA MAJOR INFECTION IN MAMMALS, which is incorporated herein by reference in its entirety.

### GOVERNMENT RIGHTS

This invention was made with U.S. Government support under NIH Research Project Grant program NIH AI057992. The U.S. Government has certain rights in the subject matter herein.

### BACKGROUND

Oxidative degradation of the essential amino acid tryptophan to kynurenine is catalyzed by at least two structurally distinct classes of enzymes in mammals: a homeostatic enzyme, tryptophan 2,3-dioxygenase, expressed constitutively by the liver; and indoleamine 2,3-dioxygenases (IDOs), whose expression is regulated in diverse cell types by pathogen- and host-derived inflammatory signals. These inflammatory signals include the pro-inflammatory cytokines, IFN-γ and Toll-like receptor ligands (for example, lipopolysaccharide) and interactions between immune cells, for example, the engagement of costimulatory molecules on antigen presenting cells by CTLA-4. A. L. Mellor and D. H. Munn (2004) Nat. Rev. Immunol. 4(10):762-774. Two IDO homologs, termed IDO-1 and IDO-2, are known. IDO-2 was discovered recently and is also known as indoleamine 2,3-dioxygenase-like protein or proto-indoleamine 2,3-dioxygenase (IDO2, IDO-2, INDOL1 or proto-IDO). 1-methyl tryptophan (1-MT) is an available inhibitor of both IDO-1 and IDO-2.

The IDOs have been recognized as antimicrobial effectors, restricting tryptophan availability to intracellular pathogens, including tryptophan auxotrophs such as T. gondii. Accordingly, treatment of T. gondii-infected human cells with IFN-γ caused an upregulation in IDO expression and activity, leading to restriction of parasite replication. This effect on the parasites may be related to due to an IDO-induced lowering of local tryptophan concentrations, to increasing levels of bioactive tryptophan metabolites, or to both of these effects of upregulated IDO expression and activity. This restriction of the parasite replication is at least partially reversible by the addition of exogenous tryptophan. J. M. Carlin et al. (1989) J. Leukoc. Biol. 45:29; H. W. Murray et al. (1989) Infect. Immun. 57: 845-856; E. R. Pfefferkorn et al. (1984) Proc. Nat'l Acad Sci, USA 81: 908-917; J. L. Schmitz et al. (1989) Infect Immun. 57: 3254-3261.

The tryptophan auxotrophy of T. gondii provided a biological rationale for the targeting of this organism by immune-driven IDO activity. Subsequently, similar studies reported that IFN-γ induction of IDO played a role in restricting the replication of a range of intracellular pathogens, including bacterial tryptophan auxotrophs. H. W. Murray et al. (1989) Infect. Immun. 57: 845-856; J. M. Carlin et al. (1989) J. Interferon Res. 9: 329-337; L. G. Pantoja et al. (2000) Infect. Immun. 68: 6478-6489; O. Adams et al. (2004) Microbes Infect. 6:806-817; O. Adams et cd. (2004) J. Virol. 78: 2632-2341; B. Bodaghi et al. (1999) J. Immunol. 162:957-966; K. Obojes et al. (2005) J. Virol. 79: 7768-7779; M. Terajima and A. M. Leporati (2005) Viral Immunol. 18: 722-733.

Leishmania, flagellate protozoa of the family Trypansomatidae, are the pathogenic agents responsible for leishmaniasis. Like Toxoplasma, Leishmania are tryptophan auxotrophs. J. A. O'Daly and M. B. Rodriguez (1988) Acta Trop. 45:109-114. These parasites infect numerous species of mammals.

Leishmaniasis remains a significant cause of morbidity and mortality in the world at large. Available therapies are problematic due to toxicity, treatment duration and emerging drug resistance. Thus, there exists a need in the art for novel methods and compounds for the treatment of diseases caused by Leishmania protozoa.

### SUMMARY

Embodiments of the invention include methods for treating an infection of a mammal by an organism, a host immune response to which is inhibited by the activity of an indolamine 2,3-dexygenase (IDO) in the mammal. The methods can include: diagnosing, in the mammal, the mentioned infection affected by the activity of an IDO in the mammal; causing modulation in the mammal of a level of a biomolecule such as, for example, tryptophan, a bioactive tryptophan metabolite, and the like; and subsequently observing amelioration or cure of the infection as a result of the modulation.

In some embodiments, the infection can be caused by a Leishmania species. The infection can be visceral leishmaniasis or the like. Modulation of the biomolecule can include modulating activity of an enzyme such as, for example, IDO-1, IDO-2, and the like. In some embodiments, the enzyme can be inhibited, such as, for example, by administration of an effective amount of an IDO inhibitor such as, for example, 1-MT.

In various embodiments, the bioactive tryptophan metabolite can include, for example, N-formylkyneurenine, kyneurenine, quinolinate, niacin, and the like. According to the methods, the level of tryptophan in the animal can be increased. Likewise, the level of the bioactive tryptophan metabolites can be reduced.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows that IDO inhibition during acute toxoplasmosis leads to an inability to control infection. C57BL/6 mice were infected intra-peritoneally with T. gondii (ME49 strain), in the presence and absence of IDO inhibition with 1-MT. (A) Survival analysis (n=8 mice/group; closed symbols, 1-MT; open symbols, control). *P<0.0001 (Mantel-Cox test). Representative of 2 independent experiments (100% vs 0% mortality in the one not pictured). (B) Parasite burden. T. gondii cysts were counted in brain homogenates of mice sacrificed 30 d after infection. *P<0.0001 (Student's t test). (C) Serum IFN-γ. (D) Serum TNF-α. (E) Serum IL-10. Systemic cytokine production was quantified by the CCA assay 30 d after infection. F. D. Finkelman et al. (1999) Int. Immunol. 11: 1811-1817. Values shown are means±SE of 8 mice/group. (F) Kinetic analysis of parasite burden in the experiment depicted in FIG. 1A. T. gondii cysts were counted in brain homogenates of moribund (1-MT treated) mice and control mice sacrificed in parallel. (G) IL-12/23p40 mRNA. (H) IFN-γ mRNA. (I) TNF-α mRNA. (J) IL-10 mRNA. Cytokine mRNA was quantified in brain by qRT-PCR 30 d after infection. Gray bars, uninfected; black bars, white bars infected/control treatment; black bars, infected/1-MT treatment. Values shown are means±SE of data normalized for β-actin mRNA expression, of 8 mice/group (3 for uninfected mice). *P<0.005 (Student's t test), compared with control treatment.

FIG. 2 shows that IDO inhibition during latent infection with T. gondii induces disease reactivation. (A) Survival analysis (n=8 mice/group). 1-MT was begun 30 d after infection (dotted line). (B) Parasite burden. T. gondii cysts were counted in brain homogenates of mice sacrificed at the times indicated. (C) Serum IFN-γ. (D) Serum TNF-α. (E) Serum IL-10. Systemic cytokine production was quantified by the CCA assay at the times indicated; values shown are means+SE of 5-6 mice/group. Closed symbols; 1-MT; open symbols, control. *P<0.01, **P<0.05. The effect of IDO inhibition during latent T. gondii infection on brain cytokine mRNA expression: (F) IL-12/23p40 mRNA. (G) IFN-γ mRNA. (H) TNF-α mRNA. (I) IL-10 mRNA. Cytokine mRNA was quantified in brain by qRT-PCR at the times indicated. Gray bars, uninfected; black bars, white bars, infected/control treatment; black bars, infected/1-MT treatment. Values shown are means±SE of data normalized for p-actin mRNA expression, of 6 mice/group (3 for uninfected mice). *P<0.01, compared with control treatment.

FIG. 3 shows IDO expression during toxoplasmosis. (A) IDO-1 mRNA. (B) IDO-2 mRNA. mRNA was quantified in brain by qRT-PCR 30 d after infection. Gray bars, uninfected; black bars, white bars, infected/control treatment; black bars, infected/1-MT treatment. Values shown are means±SE, of data normalized for β-actin mRNA expression, of 8 mice/group (3 for uninfected mice). *P<0.001, **P<0.05, compared with uninfected mice. IDO-1 and -2 mRNA expression in the brain was similarly elevated 40 d and 70 d after infection.

FIG. 4 shows that IDO inhibition during cutaneous leishmaniasis leads to enhanced parasite clearance, associated with decreased immune counter-regulation. C57BL/6 mice were infected intradermally with L major (V1 strain), in the presence and absence of IDO inhibition with 1-MT. (A) Lesion size, n=36 mice/group to begin with (with subsequent harvest of 6 mice/group at 1, 2, 4, 6, 9, and 12 wk after infection). Closed symbols, 1-MT; open symbols, control. *P<0.001 (2-way ANOVA). (B) Parasite burden. Quantification of parasite outgrowth from titrations of ear tissue lysates was performed at the indicated times. Values shown are means+SE of 6 mice/group. Closed symbols, 1-MT; open symbols, control. *P<0.05. (C) Serum IFN-γ (D) Serum IL-10. Systemic cytokine production was quantified by the CCA assay 4 wk after infection. (G) Lesional effector/regulatory T cell ratio. Effector (TCR⁺CD4⁺CD25⁺FOXP3−) and regulatory (TCR⁺CD4⁺FOXP3⁺) T cells were quantified by flow cytometry 4 wk after infection. Values shown (C-D, G) are means±SE of 6 mice/group. *P<0.001, **P<0.01, *P<0.05. The effect of IDO inhibition during cutaneous leishmaniasis on antigen-specific cytokine production. (E) IFN-γ. (F) IL-10. Cytokines were quantified by ELISA in the supernatants of soluble L. major antigen-stimulated cells isolated from draining lymph nodes 4 wk after infection. Values shown are means+SE of 6 mice/group. *P<0.05. (G) Lesional effector/regulatory T cell ratio.

### DESCRIPTION

Definitions

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russell, Molecular Cloning: A laboratory Manual 3^{rd} ed., Cold Spring Harbor Laboratory Press (Cold Spring Harbor, N.Y. 2001), provide one skilled in the art with a general guide to many terms used in the present application.

As used herein, the term “treating” or “treatment” with respect to disease, in some embodiments, refers to preventing the disease, for example, causing the clinical symptoms of the disease not to develop in an animal that is exposed to or predisposed to the disease, but does not yet experience or display symptoms of the disease. In some embodiments, the term refers to inhibiting the disease, for example, arresting the development of the disease or its clinical symptoms. In some embodiments, the term refers to relieving the disease, completely or partially, for example, causing regression of the disease or its clinical symptoms.

As used herein, the term “modulating” tryptophan, and grammatical equivalents, refers to an agent(s) that, in some embodiments, is capable of affecting directly the concentration of tryptophan or of bioactive tryptophan metabolites such as N-formylkynurenine, kyneurenine, quinolinate, or niacin. In other embodiments, the term refers to an agent(s) that is capable of indirectly affecting the concentration of tryptophan or of bioactive tryptophan metabolites. In some embodiments, the presence of the agent(s) results in a decrease in the concentration of tryptophan relative to the concentration of tryptophan in the absence of the agent(s). A “decrease” in concentration is witnessed if there is a lower observed tryptophan level of at least 2%, 5%, 10%, 15%, 25%, 35%, 45%, 55%, 65%, 75%, 85%, 95% or more, in the presence of the agent(s) relative to that observed in the absence of the agent(s). In some embodiments, the presence of the agent(s) results in an increase in the concentration of tryptophan relative to the concentration of tryptophan in the absence of the agent(s). An “increase” in concentration is witnessed if there is an increase in the level of observed bioactive tryptophan metabolites of at least 2%, 5%, 10%, 15%, 25%, 35%, 45%, 55%, 65%, 75%, 85%, 95% or more, in the presence of the agent(s) relative to that observed in the absence of the agent(s).

As used herein, the term “effective amount” refers to, in some embodiments, an amount of an agent, for example, a therapeutic agent sufficient to result in the amelioration of one or more symptoms of a disorder. In other embodiments, a therapeutically effective amount refers to an amount of therapy, for example, a therapeutic agent sufficient to prevent advancement of a disorder. In other embodiments, a therapeutically effective amount refers to an amount of therapy, for example, a therapeutic agent sufficient to cause regression of a disorder. In other embodiments, a therapeutically effective amount refers to an amount of a therapy, for example, a therapeutic agent sufficient to enhance or improve the therapeutic effect(s) of another therapy.

As used herein, the term “inhibiting,” and grammatical equivalents, refers a reduction in biological activity. A reduction in the biological activity can include an decrease of 5%, 10%, 15%, 25%, 35%, 45%, 55%, 65%, 75%, 85%, or 95% or more, or 1.5-fold, 2-fold, 5-fold, 10-fold, 25-fold, 50-fold, 100-fold or more, of a measured biological activity in the presence of an agent(s) relative to its absence.

One skilled in the art will recognize many methods and materials similar or equivalent to those described herein that can be used in the practice of the present subject matter. Indeed, the present subject matter is in no way limited to the methods and materials described.

The instant disclosure relates to the pathogen-specific, antagonistic or counter-regulatory roles of IDO enzymes during infection. In various organisms, these enzymes serve as, for example, anti-microbial effectors, facilitators of microbial clearance, and immune regulators, suppressing the anti-microbial immune response and microbial clearance. In particular, the present disclosure relates to the predominantly counter-regulatory role that IDO-1 and IDO-2 play in certain infectious diseases, more specifically in leishmaniasis, and even more specifically in visceral leishmaniasis.

Leishmania and leishmaniasis

Leishmania cause a wide spectrum of human disease. See Herwaldt, B. L. 1999. Leishmaniasis. Lancet 354:1191-9. At the severe end of the spectrum, visceral leishmaniasis (kala azar), due to disseminated parasitism of macrophages and dendritic cells, causes an annual mortality of approximately 50,000, largely in India and Sudan. See Reithinger, R. 2008. Leishmaniases' burden of disease: ways forward for getting from speculation to reality. PLoS Negl Trop Dis 2:e285. Kala azar has also emerged as a significant problem in the setting of HIV/AIDS, visceral leishmaniasis being the second most common opportunistic tissue protozoal disease (after toxoplasmosis) in people infected with HIV. See Karp, C. L., and F. A. Neva. 1999. Tropical infectious diseases in human immunodeficiency virus-infected patients. Clin Infect Dis 28:947-63; quiz 964-5. Available therapies for kala azar, including pentavalent antimonials, amphotericin B preparations, miltefosine and paromomycin, are problematic due to emerging drug resistance, toxicity, need for lengthy treatment and/or the development of post-kala azar dermal lesions. See Croft, S. L., S. Sundar, and A. H. Fairlamb. 2006. Drug resistance in leishmaniasis. Clin Microbiol Rev 19:111-26; Olliaro, P. L., P. J. Guerin, S. Gerstl, A. A. Haaskjold, J. A. Rottingen, and S. Sundar. 2005. Treatment options for visceral leishmaniasis: a systematic review of clinical studies done in India, 1980-2004. Lancet Infect Dis 5:763-74; Pandey, B. D., K. Pandey, O. Kaneko, T. Yanagi, and K. Hirayama. 2009. Relapse of visceral leishmaniasis after miltefosine treatment in a Nepalese patient. Am J Trop Med Hyg 80:580-2; Sundar, S., D. K. More, M. K. Singh, V. P. Singh, S. Sharma, A. Makharia, P. C. Kumar, and H. W. Murray. 2000. Failure of pentavalent antimony in visceral leishmaniasis in India: report from the center of the Indian epidemic. Clin Infect Dis 31:1104-7; Velez, I. D., L. M. Colmenares, and C. A. Munoz. 2009. Two cases of visceral leishmaniasis in Colombia resistant to meglumine antimonial treatment. Rev Inst Med Trop Sao Paulo 51:231-6. There is thus a clear need for novel therapeutic approaches to this neglected tropical disease.

Many mammals are potential host reservoirs, including rodents, foxes, and jackals, tree sloths, and dogs. Close human interactions with domesticated dogs are believed to be a significant source of human infection. Vanloubbeeck and Jones (2004) Ann. NY. Acad. Sci. 1026:267-272.

Nineteen species of Leishmania are potentially capable of infecting humans, and depending on the species of Leishmania involved and factors peculiar to the host (genetic, immunological, etc.), they are the source of very diverse clinical manifestations. Some of the most significant include L. major, L. infantum, L. donovani, L. mexicana, L. hraziliensis, L. chagasi, and L. amazonensis (WHO (2005) Leishmaniasis Home).

Like Trypanosoma brucei and cruzi, Leishmania are highly adaptive and have several life stages. Leishmania can exist in two forms: a mobile flagellated form called a promastigote, and a smaller non-mobile, non-flagellated intracellular form, the amastigote. The promastigotes are found in the gut of the sandfly, while amastigotes infect humans and other vertebrate hosts. The parasite is transmitted by the bite of a sandfly. The sandfly is difficult vector to control.

Within the insect, amastigotes transform in to the promastigote form. The promastigotes then migrate to the midgut of the fly, where they live extracellularly and multiply by binary fission. Promastigotes then move forward to the esophagus and the salivary glands of the insect. When the sandfly next feeds on a mammalian host, the Leishmania promastigotes are transferred to the host.

Once in the host, the promastigotes are taken up by the macrophages where they revert to the amastigote form. Amastigotes multiply inside the macrophages, eventually leading to the lysis of the macrophages. Vanloubbeeck and Jones (2004) Ann. NY. Acad. Sci. 1026:267-72. The released amastigotes are taken up by additional macrophages and so the cycle continues. Ultimately all the organs containing macrophages and phagocytes are infected, especially the spleen, liver and bone marrow (WHO (2005) Leishmaniasis Home).

Leishmaniasis develops mainly into three distinct clinical forms: cutaneous, mucocutaneous, and visceral depending on whether the parasites affect the mononuclear phagocytic system of the dermis, the mucous membranes, or the internal organs. The cutaneous lesion can remain localized at the point of inoculation of the parasite and correspond to a benign form with spontaneous healing. Besides this form, more serious pathologies exist, caused by disseminated cutaneous leishmaniasis and mucocutaneous leishmaniasis, which are very mutilating and disfiguring.

The most serious, and often fatal if untreated is visceral leishmaniasis (kala azar), with symptoms including fever, malaise, weight loss, anemia, swelling of the spleen, liver and lymph nodes. Primarily affected organs are the liver, spleen, bone marrow and other elements of the reticuloendothelial system, which are enlarged due to the infected macrophages. After a few months to a year, the patient becomes emaciated and exhausted. Death is generally due to other concurrent infections. The visceral form of leishmaniasis can also incubate for months or years before becoming clinically apparent. Furthermore, the disease can manifest itself in immunocompromised patients years after exposure in endemic regions. Because of the reduced ability of such patients to resist disease, treatment modalities for this form of the disease are most urgently required.

The most common manifestation is cutaneous leishmaniasis, resulting in multiple skin lesions and scarring. Mucocutaneous leishmaniasis begins with skin ulcers that spread and cause massive tissue destruction, especially of the nose and mouth and leaves victims horribly disfigured. Vanloubbeeck and Jones (2004) Ann. N.Y. Acad. Sci. 1026:267-72.

Drug therapy is limited to a few highly toxic compounds (Sundar & Rai (2002) Curr. Opin. Infect. Dis. 15(6):593-8), and evidence of drug resistance has narrowed an already small pool of candidate drugs. Murray (2004) Am. J. Trop. Med. Hyg. 71(6):787-94.

### Role of Tryptophan and IDO

Tryptophan (Trp) is an essential amino acid required for the biosynthesis of proteins, niacin and the neurotransmitter 5-hydroxytryptamine (serotonin). The indoleamine 2,3-dioxygenases (also known as INDO, and including both IDO-1 and IDO-2) catalyze the first and rate limiting step in the degradation of L-tryptophan to N-formyl-kynurenine. These enzymes are structurally distinct from tryptophan dioxygenase (TDO), which is responsible for dietary tryptophan catabolism in the liver.

Interferon gamma (IFN-γ) is one of several potent inducers of IDO expression. During persistent immune activation stimulated by high levels of interferon gamma, the availability of free serum tryptophan is diminished by IDO.

In human cells, a depletion of Trp resulting from IDO activity is a prominent IFN-γ-inducible antimicrobial effector mechanism. IFN-γ stimulation induces activation of IDO, which leads to a depletion of Trp, thereby arresting the growth of Trp-dependent intracellular pathogens. IDO activity also has an antiproliferative effect on many tumor cells, and IDO induction has been observed in vivo during rejection of allogeneic tumors, indicating a possible role for this enzyme in the tumor rejection process. Daubener et al. (1999) Adv. Exp. Med. Biol., 467: 517-24; Taylor et al. (1991) FASEB J., 5: 2516-22.

Small molecule inhibitors of IDO have been developed to treat or prevent IDO-related diseases. For example, oxadiazole and other heterocyclic IDO inhibitors are reported in US 2006/0258719 and US 2007/0185165. PCT Publication WO 99/29310 reports methods for altering T cell-mediated immunity including altering local extracellular concentrations of tryptophan and tryptophan metabolites, using an inhibitor of IDO such as 1-methyl-DL-tryptophan, p-(3-benzofuranyl)-DL-alanine, p-[3-benzo(b)thienyl]-DL-alanine, and 6-nitro-L-tryptophan). Munn (1999). Reported in WO 03/087347, also published as European Patent 1501918, are methods of making antigen-presenting cells for enhancing or reducing T cell tolerance. Munn (2003). Compounds having indoleamine-2,3-dioxygenase (IDO) inhibitory activity are further reported in WO 2004/094409; and U.S. Patent Application Publication No. 2004/0234623 is directed to methods of treating a subject with a cancer or an infection by the administration of an inhibitor of indoleamine-2,3-dioxygenase in combination with other therapeutic modalities. All of the patent publications cited above are incorporated by reference, each in its entirety. Other relevant patent documents are U.S. Pat. Nos. 7,598,287 and 7,465,448.

Other IDO inhibitors include, without limitation, β-(3-benzofuranyl)-DL-alanine (Sigma-Aldrich), β-(3-benzo(b)thienyl)-DL-alanine (Sigma-Aldrich), 6-nitro-L-tryptophan (Sigma-Aldrich), indole 3-carbinol (LKT Laboratories; St. Paul, Minn.), 3,3′-diindolylmethane (LKT Laboratories), epigallocatechin gallate (LKT Laboratories), 5-Br-4-Cl-indoxyl 1,3-diacetate (Sigma-Aldrich), 9-vinylcarbazole (Sigma-Aldrich), acemetacin (Sigma-Aldrich), 5-bromo-DL-tryptophan (Sigma-Aldrich), 5-bromoindoxyl diacetate (Sigma-Aldrich), phenyl-TH-DL-trp (3-(N-phenyl-thiohydantoin)-indole) (Sigma-Aldrich), propenyl-TH-DL-trp (3-(N-allyl-thiohydantoin)-indole) (Asinex; Moscow, Russia), methyl-TH-DL-trp (3-(N-methyl-thiohydantoin)-indole) (Sigma-Aldrich), brassinin (LKT Laboratories), 5-methyl-brassinin (Mehta, et al. (1994) Anticancer Res., 14:1209-1213); 3,3′-diindolylmethane (DIM; LKT Laboratories), indole-3-carbinol (I3C; LKT Laboratories), and the IDO inhibitors provided in U.S. patent application Ser. No. 10/550,444. IDO inhibitors can selectively or preferentially inhibit IDO1 and/or IDO2. IDO inhibitors can also include, without limitation, nucleic acid molecules for example, siRNA, antisense oligonucleotides, peptides, chemical compounds, and antibodies, or biologically active fragments thereof.

Expected Response of a Tryptophan Auxotroph to IDO Inhibition: T. gondii

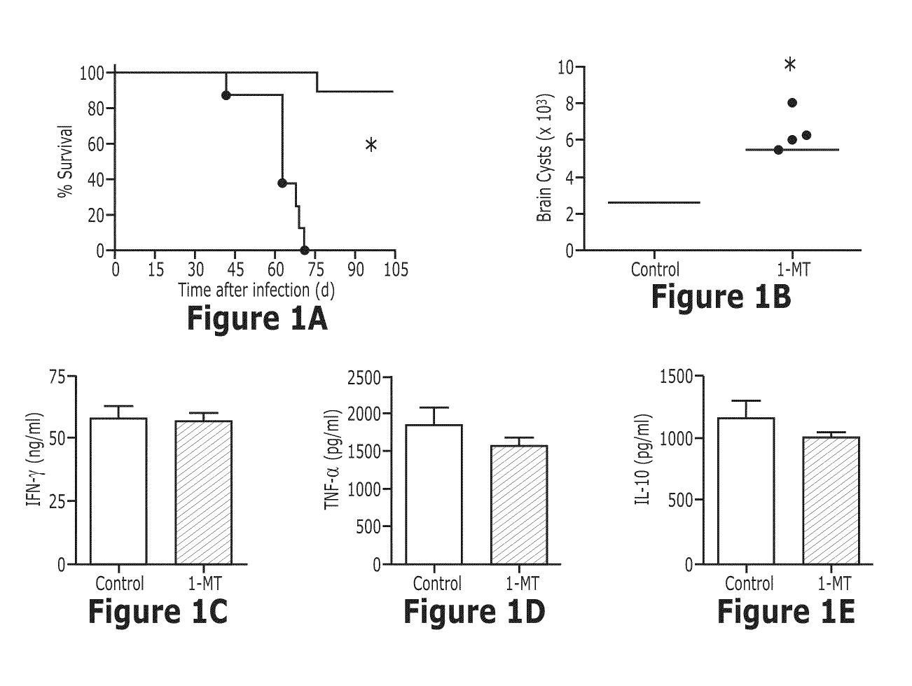

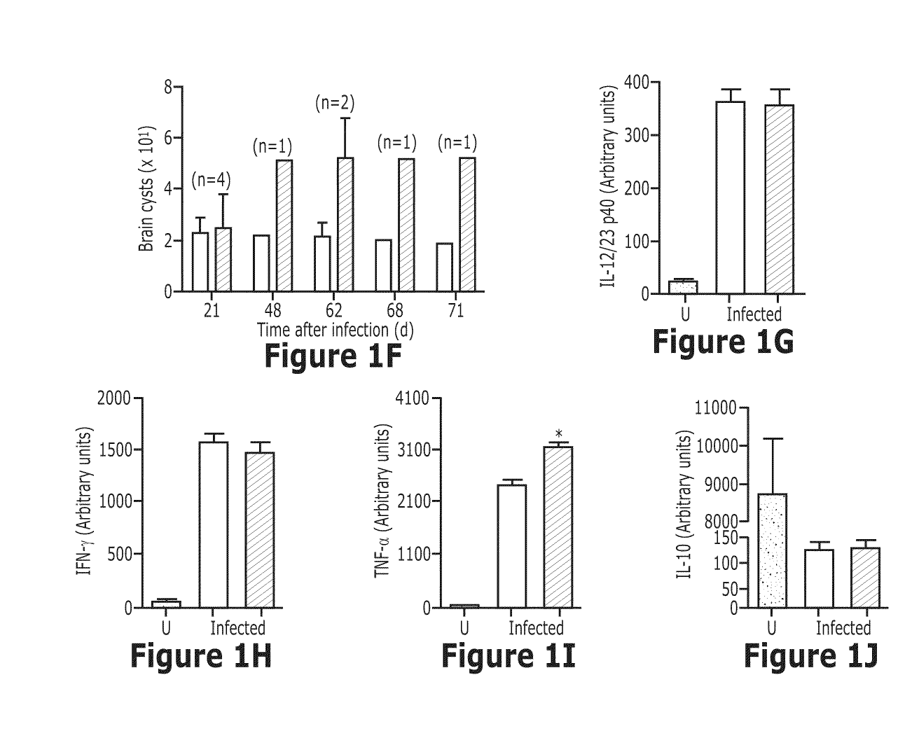

The action of 1-MT and other IDO inhibitors to increase the amount of available tryptophan and reduce levels of bioactive tryptophan metabolites, thereby promoting the growth of parasitic tryptophan auxotrophs, leading to a failure to control the parasite or the immune response to the parasite, and worsening the associated disease state in infected hosts, is paradigmatically illustrated by the case of T. gondii. As will be apparent from the results described below, this organism responds to inhibition of IDO and concomitant increased availability of tryptophan as would be expected: it proliferates, and the associated disease state of the host worsens. Specifically, the survival of mice infected with T. gondii, and provided with the IDO inhibitor 1-methyl-tryptophan (1-MT), was compared to mice not receiving 1-MT. Since Trp depletion arrests the growth of T. gondii, it was predicted that inhibition of IDO, with concomitant increase in Trp levels and reduction in bioactive tryptophan metabolites, would lead to increased mortality. This prediction was borne out as shown in FIG. 1A. Inhibition of IDO during murine toxoplasmosis led to 100% mortality with the majority of mice abruptly succumbing between 60-75 days post infection.

To evaluate parasite load, infected animals were killed and the number of T. gondii cysts present in brain homogenates from these animals were counted. As shown in FIG. 1B, it was found that the number of cysts in mice given 1-MT was significantly higher than mice not given 1-MT. Examination of brain homogenates of mice sacrificed at various time points after infection revealed that whereas the observed parasite burden was nearly the same between mice provided with 1-MT compared to those not at 21 days post infection, the parasite burden in mice treated with 1-MT was found to be much higher at subsequent time points, as shown in FIG. 1C. Therefore, IDO inhibition is associated with significantly increased parasite burdens.

An immunoregulatory role of Trp depletion has received much attention recently. Several lines of evidence suggest that IDO is involved in induction of immune tolerance. Studies of mammalian pregnancy, tumor resistance, chronic infections and autoimmune diseases have shown that cells expressing IDO can suppress T-cell responses and promote tolerance. Pathophysiological roles for IDO-mediated immunosuppression have also been described.

Inhibition of allogeneic T cell responses by IDO-expressing trophoblast cells is thought to facilitate maternal/fetal tolerance. A. L. Mellor and D. H. Munn (2004) Nat. Rev. Immunol. 4: 762-774. The role of IDO1 in maternal immunosuppression has been demonstrated further by the ability of 1-methyl-tryptophan (1MT), a specific and bioactive IDO1 inhibitor (Cady and Sono (1991) Arch. Biochem. Biophys. 291:326-333), to elicit MHC-restricted and T cell-mediated rejection of allogeneic mouse concepti. Mellor et al. (2001) Nat. Immunol. 2:64-68; Munn et al. (1998) Science 281: 1191-93. This effect is consistent with the high levels of IDO1 expression in placental trophoblast cells. Sedlmayr et al. (2002) Mol. Hum. Reprod. 8:385-391.

IDO is expressed by a high frequency of dendritic cells in tumor-draining lymph nodes, as well as by many tumors, and IDO inhibition can rescue anergic, tumor antigen-specific T cell effector function, inhibiting tumor growth in mouse models. D. H. Munn et al. (2004) J. Clin. Invest. 114: 280-; C. Uyttenhove et al. (2003) Nat. Med. 9: 1269-.

Increased levels of IFNs and elevated levels of urinary Trp metabolites have been observed in autoimmune diseases. It has been postulated that systemic or local depletion of Trp occurring in autoimmune diseases can relate to the degeneration and wasting symptoms of these diseases. In support, high levels of IDO were observed in cells isolated from the synovia of arthritic joints.

In immunocompetent animals infected with T. gondii, serious disease is averted by a vigorous immune response, dominated by IFN-γ production, which induces parasite killing and the transformation of remaining parasites into the dormant (bradyzoite; cyst) form, largely in the central nervous system. G. S. Yap et al. (2006) Microbes Infect. 8(4):1174-1178.

This systemic immune response is kept under tight control. Neither genetic deficiency of mediators driving the protective immune response, for example, IFN-γ, nor of mediators important for restraining the response for example, IL-10, is compatible with survival during toxoplasmosis. G. S. Yap et al. (2006) Microbes Infect. 8(4):1174-1178. Mice with the former deficiency die with increased parasite burdens; with the latter, of unrestrained production of pro-inflammatory mediators and decreased parasite burdens. G. S. Yap et al. (2006) Microbes Infect. 8(4):1174-1178. Thus, the mortality observed with IDO inhibition during toxoplasmosis is compatible with an inability to control either the parasite or the immune response to the parasite. Thus, IDO is a key antimicrobial effector during toxoplasmosis.

To address whether IDO inhibition effects the immune response to T. gondii infection, the serum levels of the pro- and anti-inflammatory cytokines, IFN-γ, TNF-α, and IL-10 was analyzed as shown in FIGS. 1D-F. No significant alteration in the levels of these cytokines was witnessed. A result largely mirrored by a lack of biologically important changes in the expression levels of mRNA for IL-12/23p40, IFN-γ, TNF-α and IL-10 in the central nervous system FIGS. 1G-J. Thus, IDO inhibition throughout the course of toxoplasmosis is associated with a late failure to control parasite replication, in the absence of significant effects on the inflammatory response.

Based on the survival rate data (FIG. 1A), death of 1-MT treated mice did not occur during early infection, but at a time when latent infection has been normally achieved in wild type mice in this model. T. M. Scharton-Kersten et al. (1997). J. Exp. Med. 185(7): 1261-1274; D. Schluter et al. (1999) J. Immunol. 162(6): 3512-3518. This appears to be similar to previous reports in NOS2-deficient mice.

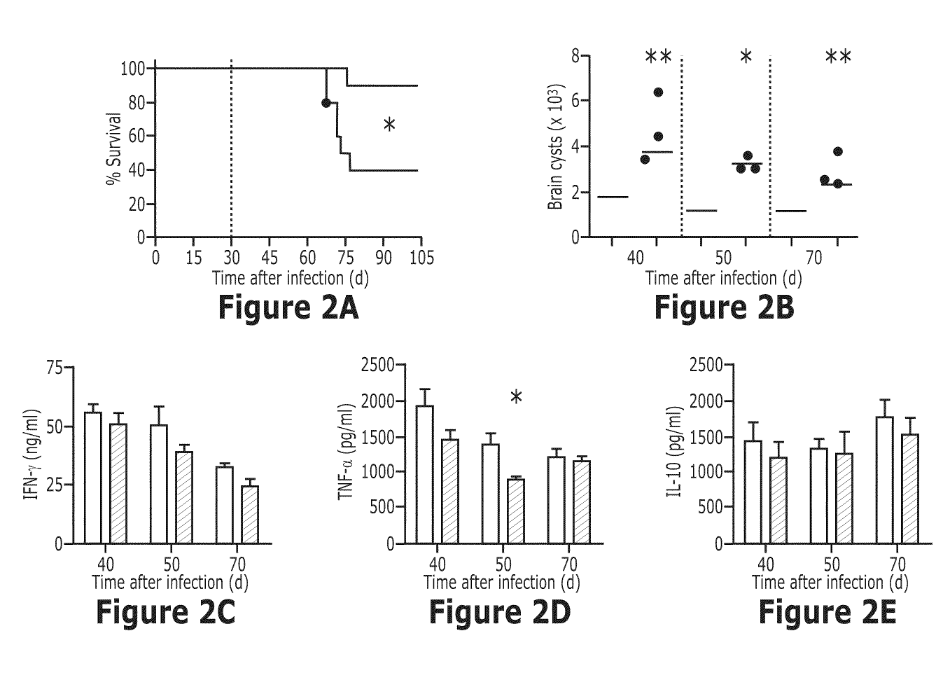

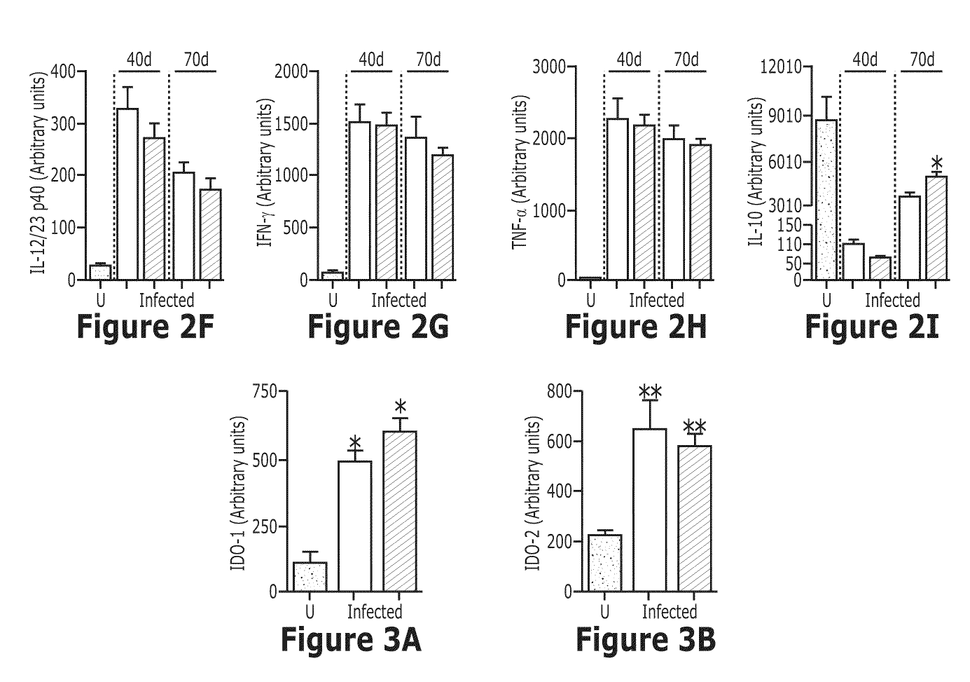

In order to analyze the effect of IDO inhibition on latent T. gondii infection, mice were treated with 1-MT beginning 30 days after infection. IDO inhibition led to reactivation of disease and increased mortality (FIG. 2A). This was associated with increased parasite burdens as evidenced by increased numbers of cysts found in brain homogenates (FIG. 2B). Thus, IDO is important for the maintenance of latent infection with T. gondii. An analysis of the levels of local and systemic production of inflammatory mediators revealed relatively little differences between infected mice treated with 1-MT and those not throughout the course of the experiment. (FIG. 2C-I).

IDO1 has been proposed to modulate gene expression. This modulation is proposed to occur through a pathway involving GCN2, whose activation has been shown to lead to altered gene expression. The proposed pathway involves the following steps. First, IDO1 activity results in the metabolism of tryptophan. Second, the deprivation of tryptophan leads to tRNAs being uncharged. The presence of uncharged tRNAs results in the activation of GCN2 kinase and a general response pathway for amino acid starvation. Third, the active GCN2 kinase phosphorylates serine 52 of the alpha subunit of eukaryotic initiation factor 2 (eIF2 α), which is known to be an important translation control mechanism.

The regulation of eIF2 α activity is governed by the phosphorylation of serine 52. Currently, there are at least three known kinases, IFN-inducible dsRNA-dependent protein kinase, heme-regulated repressor, and general control (GCN2), that can phosphorylate serine 52 in eIF2 α. The phosphorylation of serine 52 in eIF2 α prevents the GDP-GTP exchange activity of eIF2 α resulting in the suppression of protein synthesis.

GCN2 has been shown to be important for IDO1-dependent responses since a GCN2 knock-out animal phenocopies the IDO1 knock-out animal.

The gene encoding IDO2 is adjacent and structurally similar to the indoleamine 2,3-dioxygenase gene and both mouse genes use multiple promoters to express transcripts with alternate 5′ exons. The IDO2 protein is expressed in the murine kidney, liver, male and female reproductive system. The two IDO enzymes can utilize a similar range of substrates, however they differ in their selectivity for some inhibitors. The selective inhibition of IDO2 by 1-methyl-d-tryptophan suggests that IDO2 activity can have a role in the inhibition of immune responses to tumors.

To examine what, if any, effects T. gondii infection and 1-MT treatment has on IDO-1 and IDO-2 expression, mRNA was isolate from the brains of uninfected mice, T. gondii infected mice and infected mice treated with 1-MT. As shown in FIGS. 3A and 3B, T. gondii infection leads to sustained upregulation of IDO-1 and -2 mRNA expression in the brain.

### Beneficial Effects of IDO Inhibition: Leishmania

The results described above obtained in hosts infected with T. gondii to which an inhibitor of IDO was administered contrast strongly with the results obtained in cases of Leishmania infection. As shown in greater detail below, administration of the IDO inhibitor 1-MT to hosts suffering from leishmaniasis had beneficial effects in inhibiting the immune system, notwithstanding the fact that the organisms of the genus Leishmania are also tryptophan auxotrophs. This is a counterintuitive and surprising result.

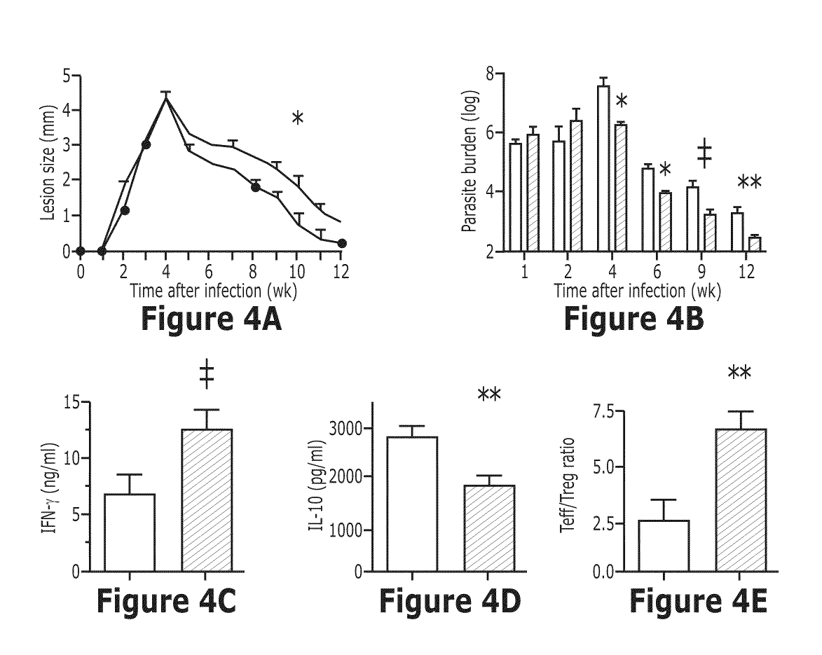

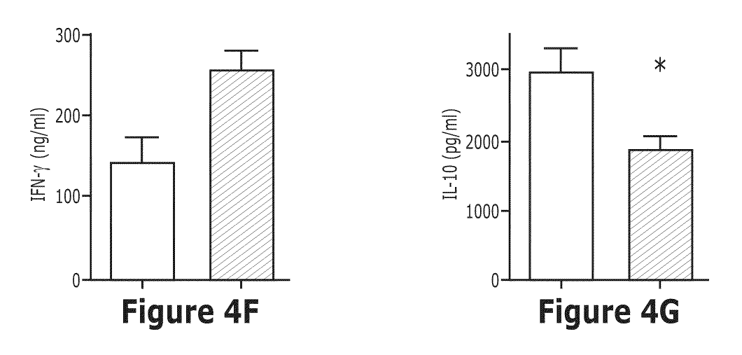

To examine the effect of 1-MT on the progression of Leishmania, the ear dermis of mice were inoculated with promastigates of L. major. Lesional size was witnessed to increase during the initial three to four weeks post infection, reaching an apex 3.5 weeks post infection (FIG. 4A). After the apex was reached, lesional size progressively decreased over the following 8 weeks. No overt differences in lesional size were apparent in mice treated with 1-MT compared to those untreated during the first weeks after infection, when lesional size was increasing. There was however, a significant difference in the size of lesions apparent in mice treated with 1-MT relative to those untreated with 1-MT after the lesional size apexed; that is, at weeks 4-12 post infection. The lesions in mice treated with 1-MT were significantly smaller than those in 1-MT untreated mice throughout the post apex period. Therefore, IDO inhibition leads to significant decreases in lesion size during the phase of parasite clearance.

Parasite burden was also examined in infected mice treated with 1-MT and compared to 1-MT untreated infected mice. As depicted in FIG. 4B, parasite burden was significantly lower in 1-MT treated mice relative to controls from weeks 4 through 12 post infection, mirroring the observed relative differences in lesional size.

In order to examine the effect of 1-MT treatment on the immune response to L. major infection, the levels of the cytokines IFN-γ and IL-10 was analyzed. During the early phase of parasite clearance, 1-MT treatment was associated with significantly increased serum IFN-γ and decreased serum IL-10 concentrations, as demonstrated in FIGS. 4C and 4D. Serum levels of IL-4 were also tested and no differences between infected mice treated with 1-MT and those not were observed (data not shown). Analysis of the cytokine production of T cells isolated from the draining lymph nodes from sites of cutaneous infection mirrored the findings observed in the serum. Relatively more IFN-γ protein was observed in infected mice treated with 1-MT relative to those untreated, while relatively less IL-10 was produced by T cells in 1-MT treated mice (FIGS. 4E and 4F).

Tryptophan catabolism by IDO functions as a counter-regulatory pathway mediating potent suppression of T cell responses in vitro and in vivo. A. L. Mellor and D. H. Munn (2004) Nat. Rev. Immunol. 4: 762-. The molecular mechanisms remain to be fully defined. Both localized tryptophan deprivation (inhibiting mTOR signaling and upregulating the GCN2 kinase stress response pathway, leading to T cell arrest and anergy) and the production of bioactive tryptophan metabolites (driving T cell apoptosis) have been implicated in various systems. Inhibition of T cell responses by IDO-expressing dendritic cells is thought to play an important physiological role in suppressing the development and expression of autoimmune and allergic diseases. A. L. Mellor and D. H. Munn (2004) Nat. Rev. Immunol. 4: 762-772.

To investigate the effects of the observed cytokine differences on T cells, the ratio of T effector relative to T regulator cells was examined (FIG. 4G). In L. major infected but 1-MT untreated mice the ratio of T effector to T regulatory cells was found to be approximately 2.5 (Teff/Treg). However, that ratio was significantly different in 1-MT treated mice, where a ratio of over 6.5 Teff/Treg was observed. Therefore, IDO inhibition is associated with a significant increase in the lesional ratio of effector to regulatory CD4⁺ T cells. Accordingly, in contrast to its role in toxoplasmosis, IDO plays a role in immune counter-regulation in Leishmaniasis, restraining both the immune response and pathogen clearance.

While appropriate caution is warranted during sustained therapeutic inhibition of IDO (for example, giving secondary chemoprophylaxis to those latently infected with T. gondii), both counter-regulatory and antimicrobial activities can provide potential novel therapeutic targets during chronic infection. For infections in which IDO plays a counter-regulatory role such as Leishmaniasis, IDO inhibition can be useful as an adjunct to anti-microbial therapy. On the other hand, for latent infections in which IDO plays an anti-microbial role, IDO can also provide a therapeutic target. Eradication of latent infection is hampered by the fact that, in the latent state, pathogens are metabolically inert and thus insensitive to the activity of current antimicrobials. For a latent pathogen against which IDO is an important anti-microbial effector mechanism, IDO inhibition can facilitate pathogen eradication through controlled reactivation, under cover of antibiotics.

While specific embodiments of the subject invention have been discussed, the above specification is illustrative and not restrictive. Many variations of the invention will become apparent to those skilled in the art upon review.

### Pharmaceutical Compositions

Another aspect of this invention is compositions that contain a safe and effective amount of a subject compound, or a pharmaceutically-acceptable salt thereof, and a pharmaceutically-acceptable carrier. As used herein, “safe and effective amount” means an amount of the subject compound sufficient to significantly induce a positive modification in the condition to be treated, but low enough to avoid serious side effects (at a reasonable benefit/risk ratio), within the scope of sound medical judgment. A safe and effective amount of the subject compound will vary with the age and physical condition of the patient being treated, the severity of the condition, the duration of the treatment, the nature of concurrent therapy, the particular pharmaceutically-acceptable carrier utilized, and like factors within the knowledge and expertise of the attending physician.

Preparing a dosage form is within the purview of the skilled artisan. Examples are provided for the skilled artisan, but are non-limiting, and it is contemplated that the skilled artisan can prepare variations of the compositions claimed.

In addition to the subject compound, the compositions of this invention contain a pharmaceutically-acceptable carrier. The term “pharmaceutically-acceptable carrier,” as used herein, means one or more compatible solid or liquid filler diluents or encapsulating substances that are suitable for administration to a mammal. The term “compatible”, as used herein, means that the components of the composition are capable of being commingled with the subject compound, and with each other, in a manner such that any interactions do not substantially reduce the pharmaceutical efficacy of the composition under ordinary use situations. Preferably when liquid dose forms are used, the compounds of the invention are soluble in the components of the composition. Pharmaceutically-acceptable carriers are, of course, of sufficiently high purity and sufficiently low toxicity to render them suitable for administration to the mammal being treated.

Some examples of substances that can serve as pharmaceutically-acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tweens®; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the subject compound is basically determined by the way the compound is to be administered. If the subject compound is to be injected, the preferred pharmaceutically-acceptable carrier is sterile, physiological saline, with a blood-compatible suspending agent, the pH of which has been adjusted to about 7.4.

If the mode of administering the subject compound is perorally, the preferred unit dosage form is therefore tablets, capsules, lozenges, chewable tablets, and the like. Such unit dosage forms contain a safe and effective amount of the subject compound, which is preferably from about 0.01 mg to about 350 mg, more preferably from about 0.1 mg to about 35 mg, based on a 70 kg person. The pharmaceutically-acceptable carrier suitable for the preparation of unit dosage forms for peroral administration are well-known in the art. Tablets typically contain conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically containe one or more solid diluents disclosed above. The selection of carrier components depends on secondary considerations like taste, cost, and shelf stability, which are not critical for the purposes of this invention, and can be readily made by a person skilled in the art.

Peroral compositions also include liquid solutions, emulsions, suspensions, and the like. The pharmaceutically-acceptable carriers suitable for preparation of such compositions are well known in the art. Such liquid oral compositions preferably containe from about 0.001% to about 5% of the subject compound, more preferably from about 0.01% to about 0.5%. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, Avicel®RC-591, tragacanth and sodium alginate; typical wetting agents include lecithin and polysorbate 80; and typical preservatives include methyl paraben and sodium benzoate. Peroral liquid compositions can also contain one or more components such as sweeteners, flavoring agents and colorants disclosed above.

Other compositions useful for attaining systemic delivery of the subject compounds include sublingual and buccal dosage forms. Such compositions typically containe one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above can also be included.

Compositions can also be used to deliver the compound to the site where activity is desired: intranasal doses for nasal decongestion, inhalants for asthma, and eye drops, gels and creams for ocular disorders.

Preferred compositions of this invention include solutions or emulsions, preferably aqueous solutions or emulsions containing a safe and effective amount of a subject compound intended for topical intranasal administration. Such compositions preferably containe from about 0.001% to about 25% of a subject compound, more preferably from about 0.01% to about 10%. Similar compositions are preferred for systemic delivery of subject compounds by the intranasal route. Compositions intended to deliver the compound systemically by intranasal dosing preferably containe similar amounts of a subject compound as are determined to be safe and effective by peroral or parenteral administration. Such compositions used for intranasal dosing also typically include safe and effective amounts of preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfate and others; aromatic agents; viscosity adjustors, such as polymers, including cellulose and derivatives thereof, and polyvinyl alcohol and acids and bases to adjust the pH of these aqueous compositions as needed. The compositions can also containe local anesthetics or other actives. These compositions can be used as sprays, mists, drops, and the like.

Other preferred compositions of this invention include aqueous solutions, suspensions, and dry powders containing a safe and effective amount of a subject compound intended for atomization and inhalation administration. Such compositions preferably containe from about 0.1% to about 50% of a subject compound, more preferably from about 1% to about 20%; of course, the amount can be altered to fit the circumstance of the patient contemplated and the package. Such compositions are typically contained in a container with attached atomizing means. Such compositions also typically include propellants such as chlorofluorocarbons 12/11 and 12/114, and more environmentally friendly fluorocarbons, or other nontoxic volatiles; solvents such as water, glycerol and ethanol, these include cosolvents as needed to solvate or suspend the active; stabilizers such as ascorbic acid, sodium metabisulfite; preservatives such as cetylpyridinium chloride and benzalkonium chloride: tonicity adjustors such as sodium chloride; buffers; and flavoring agents such as sodium saccharin. Such compositions are useful for treating respiratory disorders, such as asthma and the like.

Other preferred compositions of this invention include aqueous solutions containing a safe and effective amount of a subject compound intended for topical intraocular administration. Such compositions preferably contain from about 0.0001% to about 5% of a subject compound, more preferably from about 0.01% to about 0.5%. Such compositions also typically include one or more of preservatives, such as benzalkonium chloride, thimerosal, phenylmercuric acetate; vehicles, such as poloxamers, modified celluloses, povidone and purified water; tonicity adjustors, such as sodium chloride, mannitol and glycerin; buffers such as acetate, citrate, phosphate and borate; antioxidants such as sodium metabisulfite, butylated hydroxy toluene and acetyl cysteine; acids and bases can be used to adjust the pH of these formulations as needed.

Other preferred compositions of this invention useful for peroral administration include solids, such as tablets and capsules, and liquids, such as solutions, suspensions and emulsions (preferably in soft gelatin capsules), containing a safe and effective amount of a subject compound. Such compositions preferably containe from about 0.01 mg to about 350 mg per dose, more preferably from about 0.1 mg to about 35 mg per dose. Such compositions can be coated by conventional methods, typically with pH or time-dependent coatings, such that the subject compound is released in the gastrointestinal tract at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit® coatings, waxes and shellac.

Pharmaceutically acceptable salt(s) include but is not limited to salts of acidic or basic groups that can be present in compounds identified using the methods of the present invention. Compounds that are basic in nature are capable of forming a wide variety of salts with various inorganic and organic acids. The acids that can be used to prepare pharmaceutically acceptable acid addition salts of such basic compounds are those that form non-toxic acid addition salts, that is, salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (that is, 1,1′-methylene-bis-(2-hydroxy-3-naphthoate) salts. Compounds that include an amino moiety can form pharmaceutically or cosmetically acceptable salts with various amino acids, in addition to the acids mentioned above. Compounds that are acidic in nature are capable of forming base salts with various pharmacologically or cosmetically acceptable cations. Examples of such salts include alkali metal or alkaline earth metal salts and, particularly, calcium, magnesium, sodium lithium, zinc, potassium, and iron salts.

Any of the compositions of this invention can optionally, include other drug actives.

### Examples

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1

### Effects of IDO Inhibition on Leishmania Infection

C57BL/6 mice were infected intradermally with Leishmania major clone V1 (MHOM/IL/80/Friedlin), in the presence and absence of IDO inhibition with 1-MT. Infective-stage metacyclic promastigotes of L. major were isolated by negative selection using peanut agglutin. 10⁵ metacyclic promastigotes (in 5 μl) were inoculated intradermally into the dermis of the ear of female C57BL/6 mice using a 27.5 gauge needle. Lesion size was quantified with a vernier caliper. Parasite burden was quantified by determining the highest dilution of ear tissue lysates in which promastigote outgrowth could be detected out after 7 days of incubation. Intradermal lymphocytes were isolated from the ear by incubating ventral and dorsal dermal sheets with collagenase A, followed by filtering of fragmented tissue through a 70 μm nylon cell strainer. Cells were subsequently analyzed by flow cytometry for surface markers and intracellular FOXP3 expression. Cells isolated from lymph nodes draining lesions were incubated with L major promastigote lysates.

Cytokines were quantified by ELISA in culture supernatants harvested 48 h later. Systemic cytokine production over 18 h was quantified by the CCA assay.

Lesional mRNA expression was quantified by qRT-PCR. The following PCR primers were used:

IFN-γ
5′ TGGCTGTTTCTGGCTGTTACTG,
3′ ACGCTTATGTTGTTGCTGATGG
TNF-α
5′ CCAGACCCTCACACTCAGATCA,
3′ CACTTGGTGGTTTGCTACGAC
IL-12/23p40
5′ GGAAGCACGGCAGCAGAATA,
IL-12/23p40
3′GAACTTGAGGGAGAAGTAGGAATGG
IL-10
5′ GAAGCATGGCCCAGAAATCA, 3′ TGCTCCACTGCCTTGCTCTT
IDO-1
5′ GTGGGCAGCTTTTCAACTTC, 3′ GGGCTTTGCTCTACCACATC
IDO-2
5′TGCCTGATGGCCTATAACCAGTGT,
3′ TGCAGGATGTGAACCTCTAACGCT
β-actin
5′ GGCCCAGAGCAAGAGAGGTA, 3′ GGTTGGCCTTAGGGTTCAGG
HSV I 5′, 3′.

Results obtained indicate that adminstration of the IDO inhibitor 1-MT was beneficial to the infected mice.

### Example 2

### Treatment of Visceral Leishmaniasis

Patients present with symptoms of fever and organomegaly of long standing that is unresponsive to standard treatment, possibly with attendant anemia and pancytopenia, with a history of travel in an area in which leishmaniasis is known to be endemic. Laboratory tests confirm a diagnosis of visceral leishmaniagis. The physician begins a course of orally administered 1-MT at a dose of approximately 12.5-75 mg/kg/day. Amelioration of symptoms is observed.

### Example 3

### Treatment of Visceral Leishmaniasis

Patients present with symptoms of fever and organomegaly of long standing that is unresponsive to standard treatment, possibly with attendant anemia and pancytopenia, with a history of travel in an area in which leishmaniasis is known to be endemic. Laboratory tests confirm a diagnosis of visceral leishmaniasis. A standard course of a drug such as amphotericin is administered at normal doses, along with a course of orally administered 1-MT at a dose of approximately 12.5-75 mg/kg/day. Amelioration of symptoms is observed that is more rapid than that observed with standard drug therapy alone.

### Example 4

### Treatment of Visceral Leishmaniasis

Patients present with symptoms of fever and organomegaly of long standing that is unresponsive to standard treatment, possibly with attendant anemia and pancytopenia, with a history of travel in an area in which leishmaniasis is known to be endemic. Laboratory tests confirm a diagnosis of visceral leishmaniasis. A course of a drug such as amphotericin is administered at doses less than those usually administered, along with a course of orally administered 1-MT at a dose of approximately 12.5-75 mg/kg/day. Amelioration of symptoms is observed that is equivalent to that achieved with standard drug therapy alone.

The various methods and techniques described above provide a number of ways to carry out the invention. Of course, it is to be understood that not necessarily all objectives or advantages described need be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several advantageous features, while others specifically exclude one, another, or several features, while still others specifically mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications is herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method for treating an infection of a mammal by an organism, a host immune response to which is inhibited by the activity of an indolamine 2,3-dexygenase (IDO) in the mammal, comprising:
diagnosing, in the mammal, an infection by an organism, the host immune response to which is inhibited by the activity of an indolamine 2,3-dexygenase (IDO) in the mammal;
causing modulation in the mammal of a level of a biomolecule selected from the group consisting of tryptophan and a bioactive tryptophan metabolite; and
subsequently observing amelioration or cure of the infection as a result of the modulation.

2. A method according to claim 1, wherein said infection is caused by an organism of genus Leishmania.

3. The method according to claim 2, wherein the infection is visceral leishmaniasis.

4. A method according to claim 1, wherein modulation of the biomolecule comprises modulating activity of an enzyme selected from the group consisting of IDO-1 and IDO-2.

5. A method according to claim 4, wherein the enzyme is inhibited.

6. A method according to claim 5, wherein the enzyme is inhibited by administration of an effective amount of 1-MT.

7. The method of claim 2, wherein the bioactive tryptophan metabolite is selected from the group consisting of N-formylkynurenine, kyneurenine, quinolinate, and niacin.

8. The method of claim 4, wherein the level of tryptophan is increased.

9. The method of claim 4, wherein the level of the bioactive tryptophan metabolite is reduced.
